(19) United States
(12) Patent Application Publication
(10) Pub. No.: US 2024/0299609 A1
(43) Pub. Date: Sep. 12, 2024

(54) Bi-Voltage Air and Surface Sanitizer Device

(71) Applicant: Joshua Shane Felder, Stuart, FL (US)

(72) Inventor: Joshua Shane Felder, Stuart, FL (US)

(21) Appl. No.: 18/117,493

(22) Filed: Mar. 6, 2023

(51) Int. Cl. A61L 9/04; A61L 2/10; A61L 2/20; A61L 2/26; A61L 9/12; A61L 9/20; H01M 50/247; H02J 7/00

(52) U.S. Cl. CPC A61L 9/046; A61L 2/10; A61L 2/202; A61L 2/26; A61L 9/122; A61L 9/20; H01M 50/247; H02J 7/0048; A61L 2202/11; A61L 2202/14; A61L 2202/16; A61L 2209/111; A61L 2209/12; A61L 2209/14; A61L 2209/212; H01M 2220/30; H02J 2207/20

(57) ABSTRACT

The present disclosure provides a portable bi-voltage air and surface sanitiser device that is capable of operating using both a detachable battery module power supply and an AC power supply inlet. The device features a pair of exterior perforated panels, as well as an interior fan that efficiently draws a flow of air through the housing and across an ozone generator that converts the oxygen in the flow of air to volatile ozone particles. These particles effectively sterilise the surrounding air and neutralise foul odours and bacteria on surrounding surfaces. A distinct advantage of this device is its bi-voltage capability, which enhances its versatility and makes it suitable for use in diverse environments. The design is portable, lightweight, and easy to use, thus providing a reliable, cost-effective, and convenient solution to air and surface sanitation.

### FIELD OF INVENTION

The present invention relates generally to air and surface sanitizing devices. More specifically, the present invention relates to a device for sanitizing environments by generating ozone particles from the surrounding air to sterilize bacteria.

### BACKGROUND

In the field of air and odour removal, there are various types of devices, including activated carbon filters, photocatalytic oxidation systems, and ion generators. However, the use of ozone generators has proven to be particularly effective in neutralising odours and killing bacteria. Ozone is a potent oxidising agent that effectively breaks down organic compounds and kills bacteria and viruses.

Ozone disinfection technology was first developed by a German chemist CF Schain in 1840, and was initially used in the water treatment and disinfection industry in 1856. Since then, ozone has been widely utilised in water treatment, air purification, food processing, and other fields. With the continued development of domestic ozone disinfection technology, ozone technology has gradually made its way into many households for disinfection purposes. In household life, ozone is mainly used for air purification and disinfection, as well as sterilization of food and articles.

However, existing technologies have several drawbacks. Prior art designs in the field of air sanitising have been developed with either battery power or 110V AC power outlet configurations. Devices relying on battery power are limited in their operational time and effectiveness, as they are unable to sanitise large spaces before the battery runs out and needs to be exchanged. Conversely, devices relying on AC power are less portable and require a cable to connect to the outlet, making them unsuitable for use in vehicles or other environments where the space needs to be completely sealed. This is particularly problematic for devices that generate ozone, as ozone can be hazardous to human health.

Therefore, there is a need for a portable air sanitiser device that is capable of operating using both battery power and AC power, with the advantages of each design while mitigating their disadvantages. The present invention addresses this need by providing a bi-voltage device that enhances the versatility and portability of air sanitising technology while ensuring user safety and effectiveness.

### SUMMARY

The present disclosure provides a portable bi-voltage air and surface sanitiser device that is capable of operating using both a detachable battery module power supply and an AC power supply inlet. The device features a pair of exterior perforated panels, as well as an interior fan that efficiently draws a flow of air through the housing and across an ozone generator that converts the oxygen in the flow of air to volatile ozone particles. These particles effectively sterilise the surrounding air and neutralise foul odours and bacteria on surrounding surfaces. A distinct advantage of this device is its bi-voltage capability, which enhances its versatility and makes it suitable for use in diverse environments. The design is portable, lightweight, and easy to use, thus providing a reliable, cost-effective, and convenient solution to air and surface sanitation.

Thus, according to one aspect of the present disclosure, there is provided a portable air and surface sanitiser device, comprising: a protective housing comprising first and second panels arranged on opposing ends of the housing to each other, the panels both having a plurality of perforations or openings formed therein for allowing airflow through the housing; a bi-voltage power supply module, the power supply module comprising a power distribution unit coupled to both a detachable battery module by a DC-DC inverter and to an AC power supply inlet by an AC-DC; an electric fan coupled to the power supply module and arranged adjacent to an inner surface of the first panel; an ozone generator device coupled to the power supply module and arranged between the fan and the second panel; and a control element disposed on the housing exterior and configured to, upon actuation, cause the power supply module to send power to the electric fan and ozone generator device.

In some examples, the ozone generator device is a corona discharge device comprising a transformer for producing a high voltage across a dielectric plate.

The power supply module may be configured to cut power to the ozone generator and fan after a pre-determined time period using an internal timer.

The detachable battery module may be of a slide lock design, a stick battery insert design, or any other suitable design.

In some examples, the housing is a singular 3D-printed unit constructed such that each internal component can be bolted to a designated portion of the internal surface. This makes the manufacturing process more cost effective and is possible due to the simplicity of the design.

The housing can comprise a handle attached to an exterior top surface to make the device easier to carry.

In some examples the device can further comprise various sensors, such as but not limited to: a temperature sensor, a humidity sensor, one or more air quality sensors such as a CO2 detector and a PPM sensor. The sensors would each be coupled to an externally mounted display of the housing, showing details to the user about the surrounding air.

Other elements may also be integrated to improve usability such as an LED indicator coupled to the power supply module to show when the device is on and/or a UV lighting element coupled to the power supply module for additional sterilization of the air flowed through the housing.

A HEPA filter can also be arranged adjacent to the electric fan between the fan and the second panel within the housing to filter the air flowed through of dust particles etc.

The control element may be a single on/off rocker switch for ease of use.

### BRIEF DESCRIPTION OF THE DRAWINGS

Various embodiments of the invention are disclosed in the following detailed description and accompanying drawings.

FIG. 1A illustrates a first isometric perspective view of a first example configuration of a device according to a present disclosure.

FIG. 1B illustrates a second isometric perspective view of the first example configuration of the device.

FIG. 2A illustrates a first exploded components view of the first example configuration of the device.

FIG. 2B illustrates a second exploded components view of the first example configuration of the device.

FIG. 3 illustrates an exploded components view of a second example configuration of the device.

Common reference numerals are used throughout the figures and the detailed description to indicate like elements. One skilled in the art will readily recognize that the above figures are examples and that other architectures, modes of operation, orders of operation, and elements/functions can be provided and implemented without departing from the characteristics and features of the invention, as set forth in the claims.

### DETAILED DESCRIPTION AND PREFERRED EMBODIMENT

The following is a detailed description of exemplary embodiments to illustrate the principles of the invention. The embodiments are provided to illustrate aspects of the invention, but the invention is not limited to any embodiment. The scope of the invention encompasses numerous alternatives, modifications and equivalent; it is limited only by the claims.

Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.

### Definitions

The terminology used herein is for the purpose of describing particular embodiments only and is not intended to be limiting of the invention. As used herein, the term “and/or” includes any combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The terminology used herein is for the purpose of describing particular embodiments only and is not intended to be limiting. As used herein, the singular forms “a,” “an” and “the” are intended to include the plural forms as well, unless the context clearly indicates otherwise.

The terms “first,” “second,” and the like are used herein to describe various features or elements, but these features or elements should not be limited by these terms. These terms are only used to distinguish one feature or element from another feature or element. Thus, a first feature or element discussed below could be termed a second feature or element, and similarly, a second feature or element discussed below could be termed a first feature or element without departing from the teachings of the present disclosure.

Two example configurations are provided in the following detailed description, but it will be recognized by the reader that various aspects of the design can be changed while remaining within the scope of the present disclosure as defined by the claims.

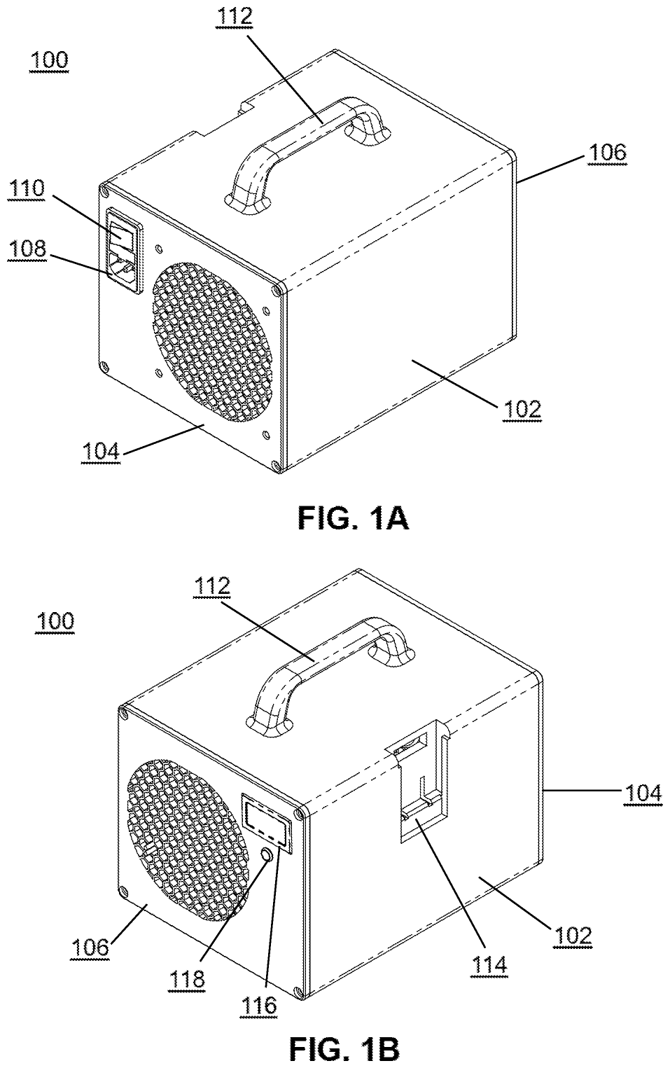

Referring to FIG. 1A and FIG. 1B, perspective views of the first example configuration of the device 100 are shown.

As can be seen, the device comprises a protective housing 102 with a front panel 104 and a rear panel 106 affixed to it, each having perforated areas that form a channel for air to flow through the housing. The panels are arranged opposite to one another for efficient airflow.

The device can operate from an AC power outlet supply, as shown by the power inlet 108 visible in FIG. 1A. A simple on/off rocker switch 110 is positioned adjacent to the inlet for activating and deactivating the device operations.

The device 100 can also operate independently from battery power, and has a port 114 for a detachable battery module to be affixed as shown in FIG. 1B. In the present example the battery port is configured to receive a stick lock battery.

The device 100 is portable, and in the present example the housing has a handle 112 arranged on the top surface of the housing to make it easier to carry. The device 100 of the present example also has a hygrometer 116 and LED indicator light 118 affixed to the opposing panel to the AC power inlet, providing information to the user on the air quality in the room and whether the device is in operation creating ozone particles.

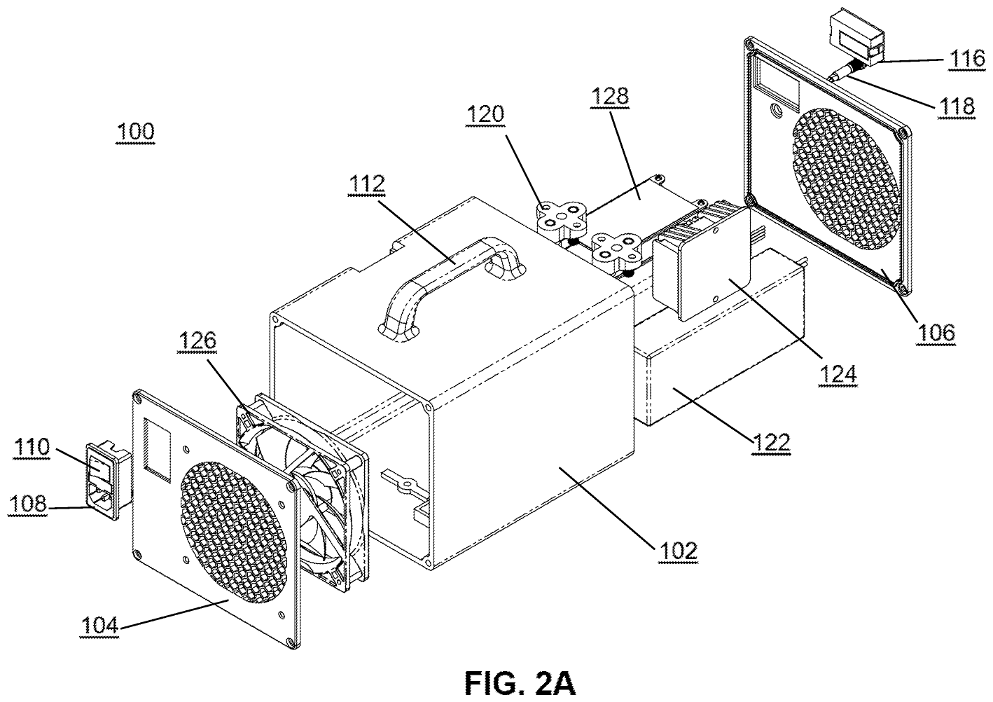

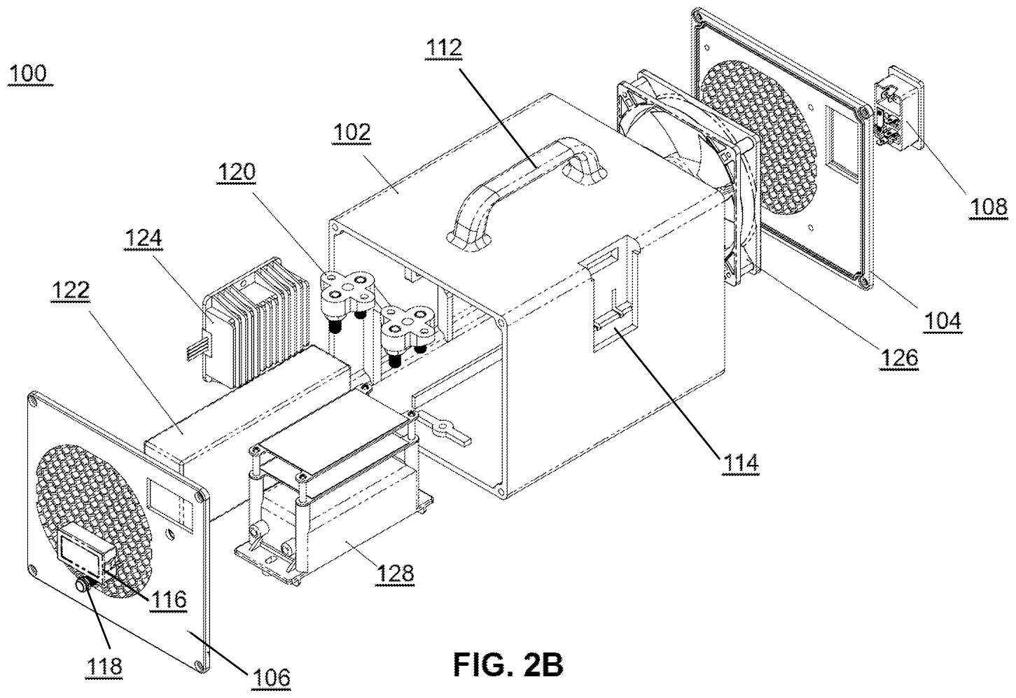

FIG. 2A and FIG. 2B illustrate exploded components views of the first example configuration of the device 100, showing the components that are normally internal to the housing.

These components include the parts necessary for the device to operate on bi-voltage sources—a pair of power distribution terminals 120. The terminals 120 are coupled to the AC power supply inlet 114 by an AC-DC inverter 122 and to the DC battery port 114 by a DC-DC inverter 124 that steps the voltage to the required levels (i.e. 12V in most cases).

Connected to this power distribution system is a DC voltage fan 126 arranged next to one of the panels 104. The fan is configured, during operation, to draw a flow of air through the housing 102.

As the air is flowed through, it passes over an ozone generator device 128 which is also coupled to the power supply. The ozone generator 128 included in the portable air sanitiser device 100 of the present example utilises a high voltage electrical discharge across a dielectric plate to convert the oxygen particles in the air flowing through the device into ozone particles. The ozone generator 128 includes a pair of metallic plates that are separated by a dielectric material, creating a capacitor-like structure. A high voltage electrical discharge is applied across the plates, creating an electrical field that ionises the oxygen molecules in the air, resulting in the formation of unstable ozone molecules. These molecules are highly reactive and unstable, and quickly react with any nearby organic compounds or bacteria, breaking them down and rendering them harmless.

The ozone particles produced by the generator 128 are mixed into the air stream flowing through the housing 102 and then circulated throughout the surrounding environment, effectively neutralising odours and killing bacteria on nearby surfaces. This process provides an efficient and effective means of sanitising the air and surfaces in various indoor and outdoor environments, such as homes, offices, vehicles, and more.

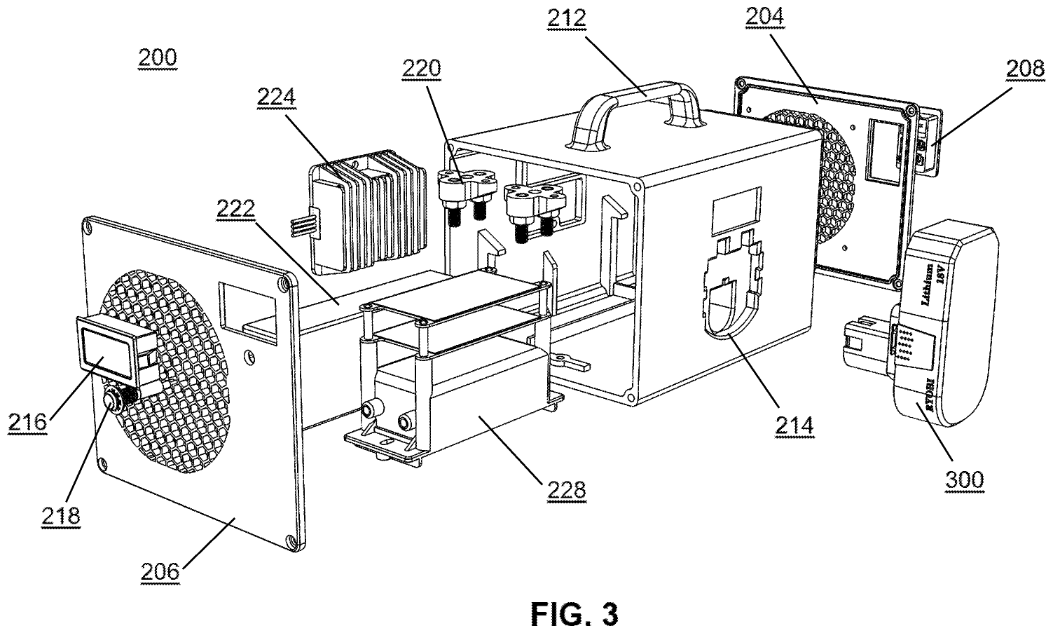

FIG. 3 illustrates an exploded components view of a second example configuration of the device 200 which uses a different type of battery port-a stick in type battery module 300 is shown ready to be connected.

The elements are the same as for the first configuration aside from that, like numerals referring to like elements.

Unless otherwise defined, all terms (including technical terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

The disclosed embodiments are illustrative, not restrictive. While specific configurations of the device have been described in a specific manner referring to the illustrated embodiments, it is understood that the present invention can be applied to a wide variety of solutions which fit within the scope and spirit of the claims. There are many alternative ways of implementing the invention.

It is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A portable air and surface sanitiser device, comprising:
a protective housing comprising first and second panels arranged on opposing ends of the housing to each other, the panels both having a plurality of perforations or openings formed therein for allowing airflow through the housing;
a bi-voltage power supply module, the power supply module comprising a power distribution unit coupled to both a detachable battery module by a DC-DC inverter and to an AC power supply inlet by an AC-DC;
an electric fan coupled to the power supply module and arranged adjacent to an inner surface of the first panel;
an ozone generator device coupled to the power supply module and arranged between the fan and the second panel; and
a control element disposed on the housing exterior and configured to, upon actuation, cause the power supply module to send power to the electric fan and ozone generator device.

2. A portable air and surface sanitiser device according to claim 1, wherein the ozone generator device is a corona discharge device comprising a transformer for producing a high voltage across a dielectric plate.

3. A portable air and surface sanitiser device according to claim 1, wherein the power supply module is configured to cut power to the ozone generator and fan after a pre-determined time period.

4. A portable air and surface sanitiser device according to claim 1, wherein the battery module is of a slide lock design.

5. A portable air and surface sanitiser device according to claim 1, wherein the battery module is of a stick battery insert design.

6. A portable air and surface sanitiser device according to claim 1, wherein the housing is a singular 3D-printed unit constructed such that each internal component can be bolted to a designated portion of the internal surface.

7. A portable air and surface sanitiser device according to claim 1, wherein the housing comprises a handle attached to an exterior top surface.

8. A portable air and surface sanitiser device according to claim 1, wherein the device further comprises a temperature sensor and display coupled to the power supply module.

9. A portable air and surface sanitiser device according to claim 1, wherein the device further comprises a humidity sensor and display coupled to the power supply module.

10. A portable air and surface sanitiser device according to claim 1, wherein the device further comprises one or more air quality sensors and a display coupled to the power supply module.

11. A portable air and surface sanitiser device according to claim 10, wherein the one or more air quality sensors include one or more of a CO2 detector and a PPM sensor.

12. A portable air and surface sanitiser device according to claim 1, wherein the device further comprises an LED indicator coupled to the power supply module.

13. A portable air and surface sanitiser device according to claim 1, wherein the device further comprises a UV lighting element coupled to the power supply module.

14. A portable air and surface sanitiser device according to claim 1, wherein the device further comprises a HEPA filter arranged adjacent to the electric fan between the fan and the second panel.

15. A portable air and surface sanitiser device according to claim 1, wherein the control element is a single on/off rocker switch.
